(19) United States
(12) Patent Application Publication
(10) Pub. No.: US 2018/0239057 A1
(43) Pub. Date: Aug. 23, 2018

(54) FORECASTING AIR QUALITY

(71) Applicant: International Business Machines Corporation, Armonk, NY (US)

(72) Inventors: Xin X. Bai, Beijing (CN); Jin Dong, Beijing (CN); Hui Du, Beijing (CN); Xiao G. Rui, Beijing (CN); Lingyun Wang, Beijing (CN); Xi Xia, Beijing (CN); Wen Jun Yin, Beijing (CN); Wei Zhao, Beijing (CN)

(21) Appl. No.: 15/439,395

(22) Filed: Feb. 22, 2017

(51) Int. Cl. G01W 1/10; G01W 1/02; G01N 33/00

(52) U.S. Cl. CPC G01W 1/10; G01N 33/0062; G01W 1/02

(57) ABSTRACT

A computer-implemented method includes comparing, by a computer processor, meteorological conditions of a first duration of time to meteorological conditions of at least one second duration of time. The beginning of the second duration of time is determined based at least in part on a first event relating to air quality conditions. The end of the second duration of time is determined based at least in part on a second event relating to air quality conditions. The method also includes outputting a forecast of air quality conditions for the first duration of time based at least in part on the comparing.

### BACKGROUND

The present invention relates in general to forecasting air quality conditions. More specifically, the present invention relates to forecasting air quality conditions by using historical air quality data and historical meteorological data.

The phrase “meteorological conditions” generally refers to environmental conditions that affect forecasting of weather conditions. Meteorological conditions can include, but are not limited to, conditions relating to temperature, air pressure, humidity, and wind speed, for example. The phrase “air quality conditions” generally refers to conditions relating to air pollution concentration and/or an amount of particulate matter. Air quality conditions can be expressed in terms of an air quality index or an air quality health index, for example. Each day can have corresponding meteorological data that measures meteorological conditions, and each day can have corresponding air quality data that measures air quality conditions.

### SUMMARY

A computer-implemented method for forecasting air quality conditions according to one or more embodiments of the present invention includes comparing, by a computer processor, meteorological conditions of a first duration of time to meteorological conditions of at least one second duration of time. The beginning of the second duration of time is determined based at least in part on a first event relating to air quality conditions. The end of the second duration of time is determined based at least in part on a second event relating to air quality conditions. The method can also include outputting a forecast of air quality conditions for the first duration of time based at least in part on the comparing.

One or more other embodiments of the present invention include a system and/or a computer program product.

### BRIEF DESCRIPTION OF THE DRAWINGS

The subject matter of the present invention is particularly pointed out and distinctly defined in the claims at the conclusion of the specification. The foregoing and other features and advantages are apparent from the following detailed description taken in conjunction with the accompanying drawings, in which:

FIG. 1 depicts an example of historical air quality data divided into time segments, in accordance with one or more embodiments of the present invention;

FIG. 2 depicts an example of matching forecasted meteorological data with segments of historical data, in accordance with one or more embodiments of the present invention;

FIG. 3 depicts an exemplary representation of matching forecasted meteorological data with different combinations of segments of historical data, in accordance with one or more embodiments of the present invention;

FIG. 4 depicts an example of a method in accordance with one or more embodiments of the present invention; and

FIG. 5 depicts an example of a computer system in accordance with one or more embodiments of the present invention.

### DETAILED DESCRIPTION

In accordance with one or more embodiments of the invention, systems, methods and computer program products for forecasting air quality are provided. Various embodiments of the present invention are described herein with reference to the drawings. Alternative embodiments can be devised without departing from the scope of this invention. References in the specification to “one embodiment,” “an embodiment,” “an example embodiment,” etc., indicate that the embodiment described can include a particular feature, structure, or characteristic, but every embodiment may or may not include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Additionally, although this disclosure includes a detailed description of a computing device configuration, implementation of the teachings recited herein are not limited to a particular type or configuration of computing device(s). Rather, embodiments of the present disclosure are capable of being implemented in conjunction with any other type or configuration of wireless or non-wireless computing devices and/or computing environments, now known or later developed.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the terms “example,” “exemplary,” and the like are used herein to mean “serving as an example, instance or illustration.” Any embodiment or design described herein as an “example,” “exemplary,” and the like are not necessarily to be construed as preferred or advantageous over other embodiments or designs. The terms “at least one” and “one or more” are understood to include any integer number greater than or equal to one, i.e. one, two, three, four, etc. The terms “a plurality” are understood to include any integer number greater than or equal to two, i.e. two, three, four, five, etc. The term “connection” is understood to include an “operable” connection, which can include an indirect “connection” and/or a direct “connection.”

For the sake of brevity, conventional techniques related to computer processing systems and computing models may or may not be described in detail herein. Moreover, it is understood that the various tasks and process steps described herein can be incorporated into a more comprehensive procedure, process or system having additional steps or functionality not described in detail herein.

Some embodiments of the present invention forecast air quality based on a combination of historical air quality data and historical meteorological data, as described in more detail below.

Each day can have meteorological data that represents a measurement of corresponding meteorological conditions, and each day can have air quality data that represents a measurement of corresponding air quality conditions. Similarly, each day in the past can have historical meteorological data and historical air quality data that represent a measurement of the corresponding past conditions.

Example approaches to weather research and forecasting include using statistical models such as: (1) an Autoregressive Moving Average (ARMA) model, (2) an Autoregressive Integrated Moving Average (ARIMA) model, (3) a Support Vector Machine (SVM), and (4) an Artificial Neural Network (ANN) model. However, such approaches to performing weather research and forecasting are not well-suited for performing air pollution forecasting.

Air quality conditions for a particular day can be affected by or related to corresponding meteorological conditions. Some contemporary methods that attempt to forecast air quality conditions are based on predicted meteorological conditions. Some such contemporary methods first attempt to predict meteorological conditions for the future duration of time. Because a user chooses the future duration of time for which air quality conditions are to be forecasted, the duration of time can be considered to be defined/pre-defined by the user. Next, such methods attempt to find a past duration of time that has historical meteorological conditions which match the predicted meteorological conditions for the future duration of time, where the past duration of time and the future duration of time are of a same length. If the methods find such a past duration of time, then the methods will forecast the air quality conditions of the future duration of time based on the historical air quality conditions of the past duration of time that was found.

For example, suppose that one uses such contemporary approaches to forecast air quality for a future time period, e.g., the next week (and thus the future time period has a pre-defined duration of seven days). Contemporary approaches first create predicted meteorological conditions for next week (i.e., over a period of seven days). Next, contemporary methods attempt to find a seven-day duration of historical meteorological conditions that matches the predicted meteorological conditions (where the predicted meteorological conditions are predicted conditions for next week). For example, suppose current methods identify a week of previous historical meteorological conditions (such as meteorological conditions between Jan. 22, 1995 through Jan. 28, 1995, for example) that matches the predicted meteorological conditions for next week. Such approaches then attempt to forecast the air quality conditions for next week, by using the historical air quality conditions that occurred between Jan. 22, 1995 and Jan. 28, 1995 as a reference.

However, the contemporary methods (such as described above) generally do not accurately forecast air quality conditions, because both air quality conditions (e.g., air pollution) and meteorological conditions vary according to natural durations/timeframes, which may not exactly match the user's pre-defined duration. For example, one may need to forecast air quality conditions for the entirety of next week (a pre-defined duration that corresponds to 1 week). However, the duration of natural patterns of air pollution/meteorological conditions may not exactly match the pre-defined (1 week) duration. For example, the entire duration of a natural pattern can last 1-2 days, or may last 1-2 months. In other words, because contemporary methods selectively sample pre-defined (e.g., one-week) durations from the historical “meteorological” data, in cases where the pre-defined duration does not match the natural durations/timeframes, contemporary methods can provide inaccurate air pollution forecasts.

Some embodiments of the present invention (examples of which are described in more detail below) analyze historical meteorological data and the historical air quality data in terms of the natural patterns of meteorological conditions and air quality. In contrast to current approaches, some embodiments of the present invention consider the natural patterns of meteorological conditions and air quality. Based at least in part on determining the natural patterns of meteorological conditions and air quality, some embodiments of the present invention refer to the determined patterns in order to forecast air pollution conditions.

As discussed above, some embodiments of the present invention identify patterns of meteorological processes that may impact air quality. Some embodiments of the present invention consider the meteorological processes that may impact air quality, by analyzing records of historical data. For example, historical air quality records can correspond to data collected over a long historical duration. Some embodiments of the present invention analyze the records of historical air quality data by dividing the historical air quality data in accordance with segments of time, where the beginning of each segment corresponds to a first event and the end of each segment corresponds to a second event. For example, each event can correspond to an air pollution event, such as a pollution accumulation event and/or a pollution dissipation event.

For example, the beginning of each segment may correspond to a pollution accumulation event, and the end of each segment may correspond to a pollution dissipation event. By way of further example, the pollution accumulation event may correspond to meteorological conditions that are conducive to pollutant concentration and the pollution dissipation event can correspond to meteorological conditions that are conducive to pollutant dispersion. By defining the beginning and end of each segment in terms of air pollution events, some embodiments of the present invention can capture natural patterns of air pollution/meteorological conditions. Because the historical air pollution accumulation events and the pollution dissipation events can occur at varied times, the identified segments can correspond to varied durations of historical time. The identified segments are not configured to be a pre-defined duration of time. Therefore, some embodiments of the present invention divide the records of historical air quality data according to a plurality of segments of time, where the duration of time corresponding to each segment begins at a first air quality event and ends at a second air quality event. In some embodiments, the pollution accumulation event can correspond to duration of time where an amount of air pollution increases, and the pollution dissipation event can correspond to a duration of time where the amount of air pollution decreases. In some embodiments, the pollution accumulation event can correspond to a point of time where pollution begins to increase, and the pollution dissipation event can correspond to a point of time where pollution begins to decrease.

In some embodiments, each identified segment may correspond to a duration of time in the past, where a pollution accumulation event and a pollution dissipation event occurred. Further, each segment may have a corresponding set of historical meteorological activity/data which reflects meteorological conditions during the time segment.

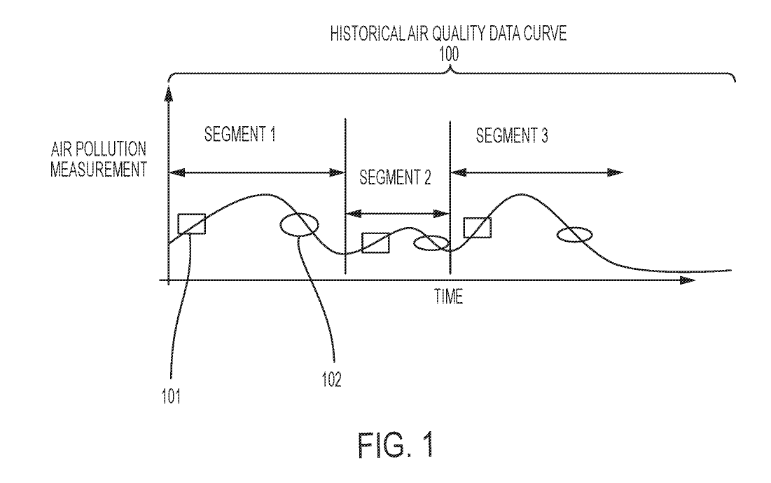

FIG. 1 depicts an example of historical air quality data divided into time segments, in accordance with one or more embodiments of the present invention. As shown in FIG. 1, air quality data can be expressed as a curve whose values vary as a function of time. Some embodiments of the present invention can determine the beginning of a segment based at least in part on the occurrence of a first air quality event. The end of a segment can be based at least in part on the occurrence of a second air quality event. Referring specifically now to the example depicted in FIG. 1, within curve 100, a first air quality event 101 can be an air pollution accumulation event, and the second air quality event 102 can be an air pollution dissipation event. In other embodiments (not depicted), the first air pollution event can be an air pollution dissipation event, and the second air pollution event can be an air pollution accumulation event. Other embodiments can have events that correspond to other types of air pollution events.

For example, referring again to FIG. 1, suppose that an analysis of historical air quality data determines that segment 1 corresponds to a duration of time between Jan. 1, 2000 and Jan. 14, 2000. In other words, the first air pollution event 101 started on January 1, and a second air pollution event 102 completed on January 14, thus defining segment 1. The meteorological data corresponding to the meteorological activity that occurred between January 1 and January 14 is the meteorological data of segment 1. In addition, suppose embodiments of the present invention determine that Segment 2 corresponds to a duration of time between Jan. 14, 2000 and Jan. 21, 2000. Referring to the example of FIG. 1, suppose embodiments of the present invention determine Segment 3. In some embodiments, the durations of the time segments can be of varied lengths.

As such, some embodiments utilize a plurality of identified segments, where each identified segment corresponds to a historical duration of time that has corresponding historical meteorological data, and the historical duration of time has a corresponding historical air pollution data, and the historical air pollution data corresponds to an air pollution pattern with an accumulation event and a dissipation event, for example. These identified segments thus have durations which are not pre-defined durations. Rather, as discussed above, by defining the beginning and end of each segment in terms of air pollution events, some embodiments of the present invention can identify segments that reflect the natural patterns of air pollution/meteorological conditions.

With the identified segments, where each segment has corresponding air pollution conditions/data and corresponding meteorological conditions/data, embodiments of the present invention can perform a matching between forecasted meteorological data and the historical meteorological data corresponding to the identified segments, as described in more detail below.

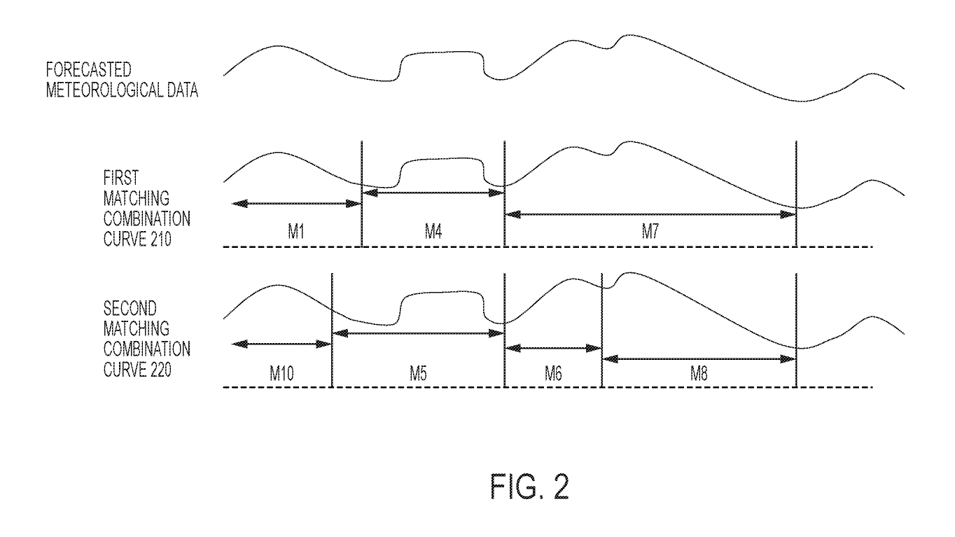

FIG. 2 depicts an example of matching forecasted meteorological data with segments of historical data, in accordance with one or more embodiments of the present invention. Some embodiments identify meteorological data that corresponds to a (pre-defined) duration of time for which a forecasted air pollution data is desired. For example, suppose that one wishes to forecast air pollution that will occur in the future i.e., between next August 1 and August 10. Some embodiments of the present invention identify meteorological data that corresponds to the duration of time between August 1 and August 10. The pre-defined duration of time for which forecasted air pollution is desired is thus a duration of 10 days. With reference now to FIG. 2, a “Forecasted Meteorological data” curve 200 can be representative of a single meteorological parameter e.g. (without limitation), a temperature curve, a humidity curve, a water vapor value curve, etc. Alternatively, curve 200 can represent a plurality and/or combination of meteorological values. Some embodiments of the present invention identify one or more segments whose corresponding historical meteorological data, when combined, matches the forecasted/predicted meteorological data between August 1 and August 10. As previously described, each segment's beginning and end can be determined based at least in part on a historical air quality event. A matching combination is generally understood as a combination of time segments whose corresponding meteorological data, when combined, matches the forecasted meteorological data.

Referring again to FIG. 2, a first matching combination curve 210 is a combination of segments whose corresponding historical meteorological data, when combined, matches forecasted meteorological data curve 200. As depicted, first matching combination curve 210 includes, at least, segment M1, segment M4, and segment M7, for example. Second matching combination curve 220 includes, at least, segment M10, M5, M6, and M8, for example. Although two possible matching combinations are shown in the example of FIG. 2, other examples may include more or less than two combinations.

In some embodiments, one or more segments whose historical meteorological data “match” the forecasted meteorological data can include segments whose corresponding meteorological data is similar to the forecasted meteorological data, within a similarity threshold. In some embodiments, a combined duration corresponding to the combined segments can “match” the duration of the forecasted meteorological data, within (another or the same) similarity threshold.

In some embodiments, possible combinations of segments whose historical meteorological data match the predicted meteorological data can be visualized as a possibility tree (also referred to below as a tree diagram).

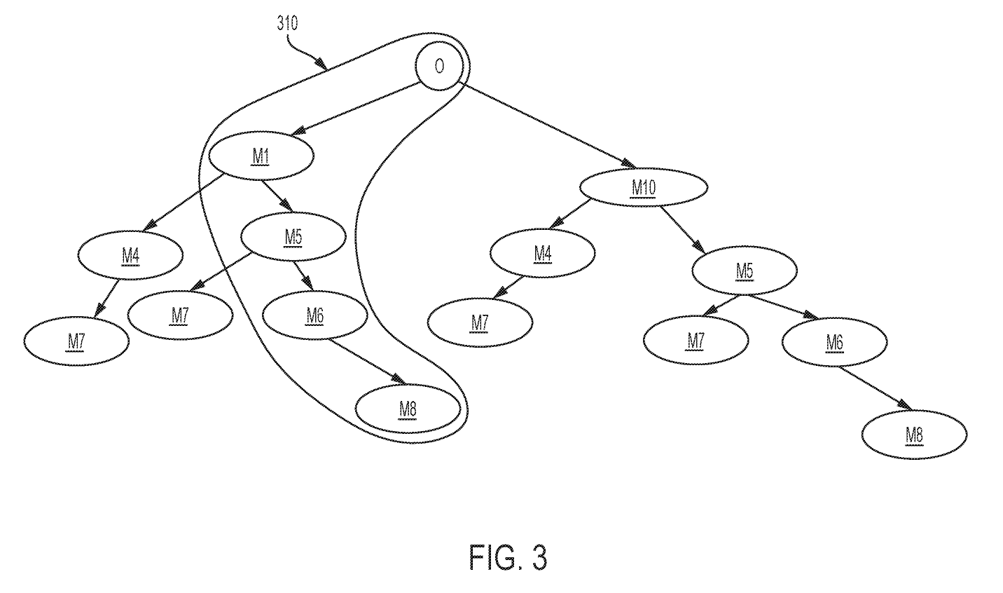

FIG. 3 depicts an exemplary representation of matching forecasted meteorological data with different combinations of segments of historical data, in accordance with one or more embodiments of the present invention. Each path within the possibility tree of FIG. 3 corresponds to a combination of segments whose historical meteorological data match the predicted meteorological data of forecasted meteorological data curve 200 of FIG. 2. For example, one combination path 310 is “M1, M5, M6, and M8,” and another combination path is “M1, M5, and M7,” and another combination path is “M1, M4, and M7,” and another combination path is “M10, M4, and M7,” etc.

Each path/combination depicted in FIG. 3 may correspond to a combination of segments that match the predicted meteorological data depicted in FIG. 2. Although two possible combinations of segments are illustrated in FIG. 2, FIG. 3 illustrates a greater number of possible combinations.

In some embodiments, each of a plurality of combinations of segments can be assigned a similarity calculation, where the similarity calculation is based (at least in part) on how closely each path/combination matches the predicted meteorological data. Some embodiments can compare each similarity calculation (of each path/combination of segments) with a threshold similarity value and if a combination's assigned similarity does not meet the threshold similarity, the combination/path is removed from further consideration. For example, each path within the tree diagram of FIG. 3 that does not satisfy a threshold similarity value can be removed from further consideration for forecasting air quality. In other words, each path within the tree diagram that does not satisfy the threshold similarity value can be removed (or “pruned”) from the tree diagram such that the remaining paths/combinations correspond to the combinations of segments whose historical meteorological data more closely match the predicted meteorological data. Such predicted meteorological data can correspond to meteorological conditions predicted to occur during the duration of time for which air quality conditions are to be forecasted. The corresponding historical air quality data of one (e.g., the most similar) or more remaining paths/combinations of segments can be used to forecast the air quality.

In some embodiments, where a plurality of remaining paths/combinations of segments is used to forecast the air quality, weightings can be applied to each remaining path where the applied weight is related to the forecasted air quality. In some embodiments, a path with an assigned similarity that more closely resembles the predicted meteorological data can be assigned a higher weight, and thus have a greater impact upon forecasting the air quality.

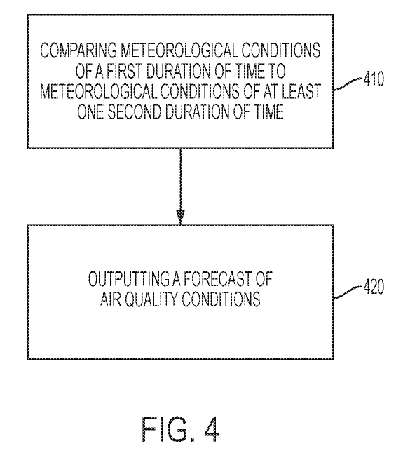

FIG. 4 depicts an example of a method in accordance with one or more embodiments of the present invention. As depicted, the computer-implemented method includes, in step 410, comparing, by a computer processor, meteorological conditions of a first duration of time to meteorological conditions of at least one second duration of time. The beginning of the second duration of time is based at least in part on a first event relating to air quality conditions. The end of the second duration of time is based at least in part on a second event relating to air quality conditions. The method includes, at 420, outputting a forecast of air quality conditions for the first duration of time based at least in part on the comparing.

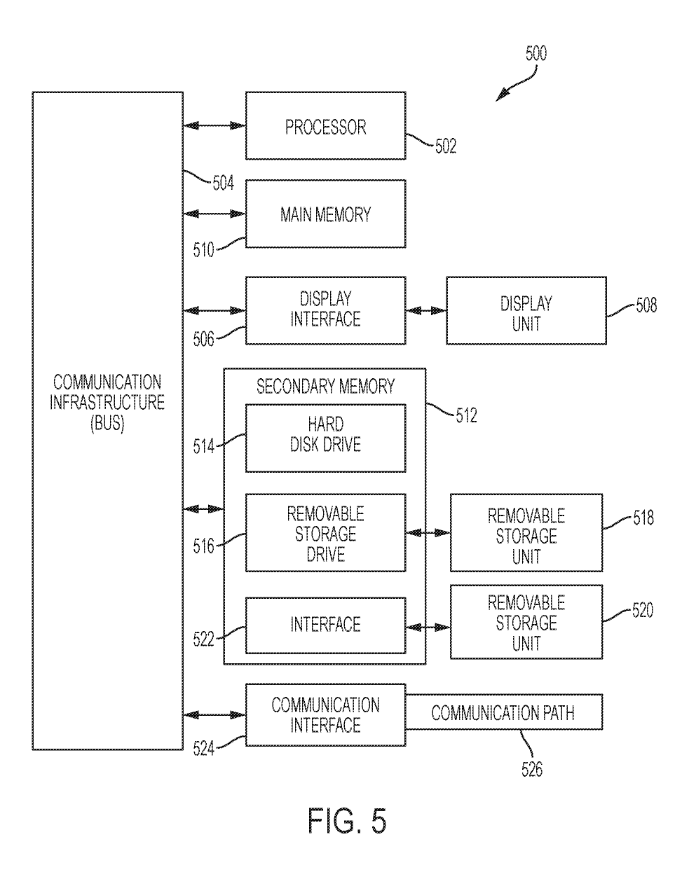

FIG. 5 depicts an example of a computer system in accordance with one or more embodiments. More specifically, some embodiments of computer system 500 of FIG. 5 implement hardware and software capable of performing one or more aspects of the air quality forecasting methods described with reference to FIGS. 1-4. Although one exemplary computer system 500 is shown in of FIG. 5, computer system 500 is depicted as including communication path 526, which can enables computer system 500 to connect with one or more other networks and/or systems (not depicted). Exemplary communications paths include (but are not limited to) wired and/or wireless communication network(s), (not depicted). Examples of such other networks and/or systems include (but are not limited to) one or more external and/or internal (e.g., intranet(s)) networks, wide area networks (WANs), local area networks (LANs), and networks of networks, such as the Internet. Computer system 500 and such other systems can be in communication via communication path 526, e.g., (without limitation) to communicate data between them.

Referring now specifically to FIG. 5, computer system 500 can include one or more processors 502. The one or more processor(s) 502 are connected to a communication infrastructure 504 (e.g., a communications bus, cross-over bar, or network). Computer system 500 can include a display interface 506 that forwards graphics, textual content, and other data from communication infrastructure 504 (or from a frame buffer not shown) for display on a display unit 508. Computer system 500 also includes a main memory 510, preferably random access memory (RAM), and can also include a secondary memory 512. Secondary memory 512 can be non-volatile, for example, a hard disk drive 514 and/or a removable storage drive 516, representing, for example, a floppy disk drive, a magnetic tape drive, or an optical disc drive. Secondary memory 512 can also be in the form of a solid state drive (SSD), a traditional magnetic disk drive, or a hybrid of the two. There also can be more than one hard disk drive 514 contained within secondary memory 512. Removable storage drive 516 reads from and/or writes to a removable storage unit 518 in a manner well known to those having ordinary skill in the art. Removable storage unit 518 represents, for example, a floppy disk, a compact disc, a magnetic tape, or an optical disc, etc. which is read by and written to by removable storage drive 516. As will be appreciated, removable storage units 518, 520 and other memory components e.g., 510, 512 can include a computer-readable medium (sometimes referred to as a computer program product) having stored therein computer software and/or data.

In some embodiments, secondary memory 512 can store and allow computer programs (also sometimes referred to as software and/or other program instructions), including software in accordance with the present invention, to be loaded into main memory 510 for execution by computer system 500. Such software can be loaded into secondary memory 512, for example, from removable storage unit 520 via interface 522. Examples of removable storage and interface include (without limitation) a program package and package interface (such as that found in video game devices), a removable memory chip (such as an EPROM, secure digital card (SD card), compact flash card (CF card), universal serial bus (USB) memory, or PROM) and associated interface socket, and other removable storage units 518, 520 and interfaces 522 which allow software and/or data to be transferred from removable storage unit 518, 520 to computer system 500.

Computer system 500 can also include a communications interface 524. Communications interface 524 allows software and data to be transferred between the computer system and external devices such as via communication path 526. Examples of communications interface 524 can include a modem, a network interface (such as an Ethernet card), a communications port, or a PC card slot and card, a universal serial bus port (USB), and the like. Software and data transferred via communications interface 524 can be in the form of signals that can be, for example, electronic, electromagnetic, optical, or other signals capable of being communicated by communications interface 524. These signals can be provided to communications interface 524 via communication path 526. Communication path 526 (sometimes referred to as a channel) carries signals and can be implemented using (without limitation) wire, cable, fiber optics, a phone line, a cellular phone link, an RF link, and/or other communications channels.

The terms “computer program medium,” “computer program product,” “computer usable medium,” and “computer-readable medium” when used herein, can refer to media such as main memory 510 and secondary memory 512, removable storage drive 516, and a hard disk installed in hard disk drive 514. Computer programs, which can include and are also sometimes called computer control logic, can be stored in main memory 510, secondary memory 512 and/or one or more removable storage units 518, 520, etc. Computer programs also can be received via communications interface 524. In general, computer programs, when run, enable processor 502 to perform the features of the computer system. Accordingly, such computer programs represent controllers of the computer system. Such computer programs, when run, can specifically enable the computer system to perform one or more features or functions of the present invention and provide corresponding technical benefits and advantages.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments described herein.

What is claimed is:

1. A computer-implemented method for forecasting air quality conditions, comprising:
comparing, by a computer processor, meteorological conditions of a first duration of time to meteorological conditions of at least one second duration of time, wherein the beginning of the second duration of time is determined based at least in part on a first event relating to air quality conditions, and the end of the second duration of time is determined based at least in part on a second event relating to air quality conditions; and
outputting a forecast of air quality conditions for the first duration of time based at least in part on the comparing.

2. The computer-implemented method of claim 1, wherein the first event comprises a pollution accumulation event, and the second event comprises a pollution dissipation event.

3. The computer-implemented method of claim 1, wherein the comparing comprises comparing a predicted meteorological data of the first duration of time with historical meteorological data of the second duration of time.

4. The computer-implemented method of claim 1, wherein the comparing comprises determining the at least one second duration of time whose historical meteorological conditions match predicted meteorological conditions of the first duration of time, and the forecast of air quality conditions for the first duration of time is determined based at least in part on air quality conditions of the matching second duration of time.

5. The computer-implemented method of claim 3, wherein the predicted meteorological data of the first duration of time corresponds to predicted meteorological conditions during the first duration of time, and the historical meteorological data of the second duration of time corresponds to historical meteorological conditions during the second duration of time.

6. The computer-implemented method of claim 4, wherein a plurality of second durations of time combine to match the predicted meteorological conditions of the first duration of time.

7. The computer-implemented method of claim 3, wherein the predicted meteorological data comprises at least one of temperature data, humidity data, water vapor data, and wind speed data.

8. A computer system comprising:
a memory, having program instructions stored therein; and
a processor communicatively coupled to the memory, wherein the program instructions are readable and executable by the processor to cause the processor to:
compare meteorological conditions of a first duration of time to meteorological conditions of at least one second duration of time, wherein the beginning of the second duration of time is determined based at least in part on a first event relating to air quality conditions, and the end of the second duration of time is determined based at least in part on a second event relating to air quality conditions; and
output a forecast of air quality conditions for the first duration of time based at least in part on the comparing.

9. The computer system of claim 8, wherein the first event comprises a pollution accumulation event, and the second event comprises a pollution dissipation event.

10. The computer system of claim 8, wherein the comparing comprises comparing a predicted meteorological data of the first duration of time with historical meteorological data of the second duration of time.

11. The computer system of claim 8, wherein the comparing comprises determining the at least one second duration of time whose historical meteorological conditions match predicted meteorological conditions of the first duration of time, and the forecast of air quality conditions for the first duration of time is determined based at least in part on air quality conditions of the matching second duration of time.

12. The computer system of claim 10, wherein the predicted meteorological data of the first duration of time corresponds to predicted meteorological conditions during the first duration of time, and the historical meteorological data of the second duration of time corresponds to historical meteorological conditions during the second duration of time.

13. The computer system of claim 11, wherein a plurality of second durations of time combine to match the predicted meteorological conditions of the first duration of time.

14. The computer system of claim 10, wherein the predicted meteorological data comprises at least one of temperature data, humidity data, water vapor data, and wind speed data.

15. A computer program product for forecasting air quality conditions, the computer program product comprising:
a computer-readable storage medium having program instructions embodied therewith, the program instructions readable by a processor to cause the processor to:
compare, by the processor, meteorological conditions of a first duration of time to meteorological conditions of at least one second duration of time, wherein the beginning of the second duration of time is determined based at least in part on a first event relating to air quality conditions, and the end of the second duration of time is determined based at least in part on a second event relating to air quality conditions; and
output a forecast of air quality conditions for the first duration of time based at least in part on the comparing.

16. The computer program product of claim 15, wherein the first event comprises a pollution accumulation event, and the second event comprises a pollution dissipation event.

17. The computer program product of claim 15, wherein the comparing comprises comparing a predicted meteorological data of the first duration of time with historical meteorological data of the second duration of time.

18. The computer program product of claim 15, wherein the comparing comprises determining the at least one second duration of time whose historical meteorological conditions match predicted meteorological conditions of the first duration of time, and the forecast of air quality conditions for the first duration of time is determined based at least in part on air quality conditions of the matching second duration of time.

19. The computer program product of claim 17, wherein the predicted meteorological data of the first duration of time corresponds to predicted meteorological conditions during the first duration of time, and the historical meteorological data of the second duration of time corresponds to historical meteorological conditions during the second duration of time.

20. The computer program product of claim 18, wherein a plurality of second durations of time combine to match the predicted meteorological conditions of the first duration of time.
